(19) United States
(12) Patent Application Publication
(10) Pub. No.: US 2020/0121604 A1
(43) Pub. Date: Apr. 23, 2020

(54) SOLID FOSMETPANTOTENATE FORMULATIONS

(71) Applicant: Retrophin, Inc., San Diego, CA (US)

(72) Inventors: Dainius Macikenas, San Diego, CA (US); Kale Ruby, Encinitas, CA (US)

(21) Appl. No.: 16/483,652

(22) PCT Filed: Feb. 7, 2018
(86) PCT No.: PCT/US2018/017266

§ 371 Date: Aug. 5, 2019

Related U.S. Application Data
(60) Provisional application No. 62/456,077, filed on Feb. 7, 2017.
(60) Provisional application No. 62/488,618, filed on Apr. 21, 2017.

(51) Int. Cl. A61K 9/16; A61K 31/664

(52) U.S. Cl. CPC A61K 9/1623; A61K 31/664; A61K 9/1652

(57) ABSTRACT

Solid formulations comprising a compound having formula (I) or a pharmaceutically acceptable salt thereof, and their use in the treatment of neurologic disorders (such as pantothenate kinase-associated neurodegeneration), are provided.

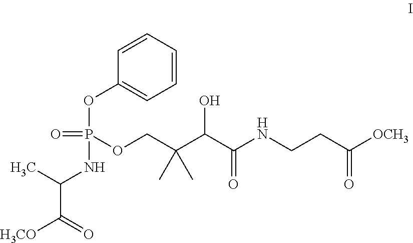 [SMILES: COC(=O)CCNC(=O)C(O)C(C)(C)COP(=O)(NC(C)C(=O)OC)Oc1ccccc1]

### BACKGROUND

### Technical Field

The present invention relates to solid formulations of a compound of formula I, including solid fosmetpantotenate formulations, and their use in the treatment of neurologic disorders (such as pantothenate kinase-associated neurodegeneration).

### Background

Pantothenate kinase-associated neurodegeneration (PKAN) is a form of neurodegeneration with brain iron accumulation (NBIA) that causes extrapyramidal dysfunction (e.g., dystonia, rigidity, choreoathetosis) (A. M. Gregory and S. J. Hayflick, “Neurodegeneration With Brain Iron Accumulation”, Orphanet Encyclopedia, September 2004). PKAN is a genetic disorder resulting from lack of the enzyme pantothenate kinase, which is responsible for the conversion of pantothenic acid (vitamin B5) to 4′-phosphopantothenic acid. 4′-Phosphopantothenic acid is subsequently converted into Coenzyme A (CoA) (as shown below) (R. Leonardi, Y.-M. Zhang, C. O. Rock, and S. Jackowski, “Coenzyme A: Back In Action”, Progress in Lipid Research, 2005, 44, 125-153).

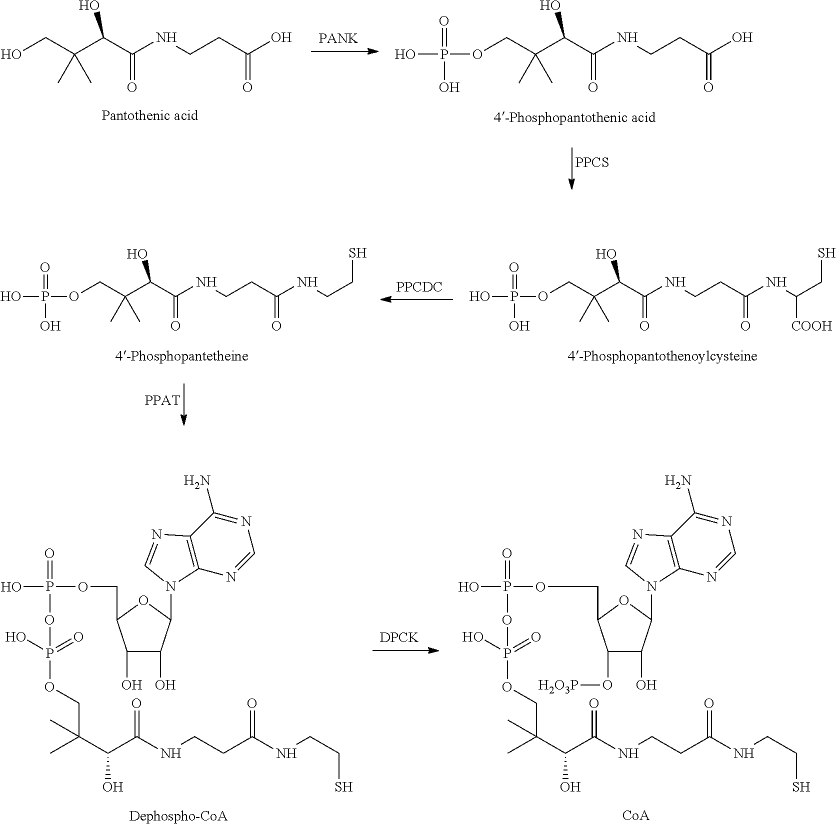

In particular, pantothenic acid is converted to 4′-phosphopantothenic acid via the enzyme pantothenate kinase (PANK), which is converted to 4′-phosphopantothenoylcysteine via the enzyme 4′-phosphopantothenoylcysteine synthase (PPCS), and subsequently decarboxylated to 4′-phosphopantetheine via 4′-phosphopantothenoylcysteine decarboxylase (PPCDC). 4′-phosphopantetheine is then appended to adenosine by the action of phosphopantetheine adenyltransferase (PPAT) to afford dephospho-CoA, which is finally converted to Coenzyme A (CoA) via dephospho-CoA kinase (DPCK).

Classic PKAN usually presents in a child's first ten to fifteen years, though there is also an atypical form that can occur up to age 40. PKAN is a progressively degenerative disease that leads to loss of musculoskeletal function with a devastating effect on quality of life. Individuals with classic PKAN often lose the ability to walk between 10 and 15 years after symptoms begin, and many require a wheelchair by their mid-teens. By this time many individuals also have difficulty chewing and swallowing, necessitating a feeding tube.

Classic PKAN is also accompanied by dystonia, a movement disorder that causes involuntary contraction and spasm of the muscles. Dystonia is typically one of the earlier symptoms to develop. Dystonia of the head or limbs is a common symptom, sometimes resulting in recurring trauma to the tongue. Extreme cases requiring complete dental extraction or involving bone fractures (caused by bone stress and osteopenia) have been known to occur. Dystonia can also cause difficulty swallowing and poor nutrition. Such secondary effects of PKAN are actually more likely to cause premature death than the neurodegenerative process.

Fosmetpantotenate is a 4′-phosphopantothenic acid prodrug in clinical development for the treatment of PKAN. However, purified fosmetpantotenate is viscous and tends to adhere to various substrates. Manufacturing pharmaceutical compositions containing such viscous, sticky substances can be challenging, as such substances tend to adhere to and accumulate on instruments used in commercial manufacturing. Use of such substances in a final pharmaceutical composition for administration to patients can also be difficult, as the tendency for the substance to stick to containers as well as instruments used for measuring and dispensing the composition may result in inaccurate or inconsistent dosing.

One potential solution for developing pharmaceutical compositions containing sticky or viscous substances is to use a liquid medium to dissolve or disperse the substance. However, liquid formulations can be costly to ship and store, and are often less stable than solid formulations (e.g., may have higher rates of chemical degradation). Additionally, liquid formulations often have characteristics patients find undesirable (e.g., bad taste, inconvenient to administer), which can reduce patient compliance.

Thus, there remains a need for improved fosmetpantotenate compositions useful for treating PKAN and other neurologic diseases associated with Coenzyme A deficiency.

### BRIEF SUMMARY

In certain aspects, the present invention is directed to solid pharmaceutical formulations comprising (a) a pharmaceutically acceptable solid excipient, and (b) a compound of formula I:

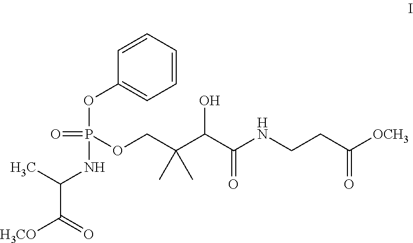 [SMILES: COC(=O)CCNC(=O)C(O)C(C)(C)COP(=O)(NC(C)C(=O)OC)Oc1ccccc1]

or a pharmaceutically acceptable salt thereof.

In certain other aspects, the present invention provides solid pharmaceutical formulations for use in treating a disorder. In one embodiment, the present invention provides formulations for use in treating a neurologic disorder. In one embodiment, the present invention provides formulations for use in treating a disorder associated with pantothenate kinase enzyme deficiency. In one embodiment, the present invention provides formulations for use in treating a subject having a disorder associated with Coenzyme A deficiency. In one embodiment, the present invention provides formulations for use in treating a condition associated with abnormal neuronal function in a subject in need thereof. In one embodiment, the present invention provides formulations for use in treating a condition associated with neuronal cell iron accumulation in a subject in need thereof. In one embodiment, the present invention provides formulations for use in treating a subject having neurodegeneration with brain iron accumulation.

In certain other aspects, the present invention provides methods of treatment comprising administering the solid pharmaceutical formulations disclosed herein to a subject. In one embodiment, a method of increasing 4′-phosphopantothenic acid production in a subject in need thereof is provided, the method comprising administering to the subject an effective amount of a formulation as disclosed herein. In another embodiment, the present disclosure provides a method of treating a subject having a disorder associated with pantothenate kinase enzyme deficiency, comprising administering to a subject in need thereof an effective amount of a formulation according to the present disclosure. In one embodiment, a method of treating a subject having a disorder associated with Coenzyme A deficiency is provided, comprising administering to a subject in need thereof an effective amount of a formulation as disclosed herein. In one embodiment, a method of treating a condition associated with abnormal neuronal function in a subject in need thereof is provided, the method comprising administering to the subject an effective amount of a formulation according to the present disclosure. In one embodiment, a method of treating a condition associated with neuronal cell iron accumulation in a subject in need thereof is provided, the method comprising administering to the subject an effective amount of a formulation according to the present disclosure. In one embodiment, a method of treating a subject having neurodegeneration with brain iron accumulation is provided, the method comprising administering to the subject an effective amount of a formulation according to the present disclosure.

These and other aspects of the present invention will become apparent upon reference to the following detailed description and attached drawings.

### BRIEF DESCRIPTION OF THE DRAWINGS

FIG. 1 shows images of solid formulations of fosmetpantotenate prepared using various drug loadings (20%, 25%, 30%, 35%, 40%, and 45% by weight) and microcrystalline cellulose as a solid excipient. 20% drug loading: free-flowing powder with no obvious appearance change compared to the microcrystalline cellulose; 25% drug loading: free-flowing powder with no obvious appearance change compared to the microcrystalline cellulose; 30% drug loading: free-flowing powder with no obvious appearance change compared to the microcrystalline cellulose; 35% drug loading: free-flowing powder with a small amount of loose agglomerates observed; 40% drug loading: flowable powder with an increased amount of loose agglomerates relative to the 35% drug loading formulation; 45% drug loading: poor flowability, and materials appeared to be wet and showed large amounts of loose agglomerates.

FIG. 2 is a graph showing the relative amount of total impurities (%) over time for various fosmetpantotenate formulations stored at 30° C. and 65% relative humidity in closed containers for 12 weeks.

FIG. 3 is a graph showing the relative amount of total impurities (excluding phenol) (as a weight %) for fosmetpantotenate formulations containing acetic acid and not containing acetic acid (“no acid”), after storage at 30° C. and 65% relative humidity in closed containers for up to 8 weeks.

FIG. 4 is a graph showing the relative amount of phenol (as a weight %) for fosmetpantotenate formulations containing acetic acid and not containing acetic acid (“no acid”), after storage at 30° C. and 65% relative humidity in closed containers for up to 8 weeks.

### DETAILED DESCRIPTION

The instant disclosure provides solid formulations comprising a compound having the following structure (I):

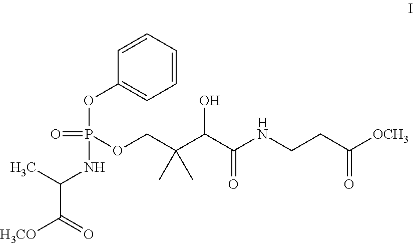 [SMILES: COC(=O)CCNC(=O)C(O)C(C)(C)COP(=O)(NC(C)C(=O)OC)Oc1ccccc1]

or a pharmaceutically acceptable salt thereof. In some embodiments, pharmaceutical compositions and methods of use are provided.

In a particular embodiment, the compound of formula I is fosmetpantotenate, or methyl 3-((2R)-2-hydroxy-4-(((((S)-1-methoxy-1-oxopropan-2-yl)amino)(phenoxy)phosphoryl)oxy)-3,3-dimethylbutanamido)propanoate.

Fosmetpantotenate has the following structure (II):

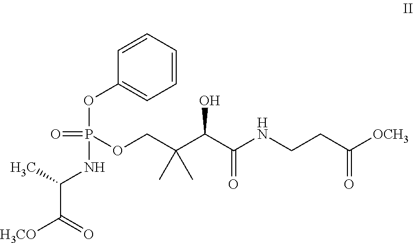 [SMILES: COC(=O)CCNC(=O)[C@H](O)C(C)(C)COP(=O)(N[C@@H](C)C(=O)OC)Oc1ccccc1]

In the following description, certain specific details are set forth in order to provide a thorough understanding of various embodiments of the invention. However, one skilled in the art will understand that the invention may be practiced without these details.

Unless defined otherwise, all technical and scientific terms used herein have the same meaning as is commonly understood by one of skill in the art to which this invention belongs. As used herein, certain items may have the following defined meanings.

Unless the context requires otherwise, throughout the present specification and claims, the word “comprise” and variations thereof, such as “comprises” and “comprising,” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the specification and claims, “including” and variants thereof, such as “include” and “includes”, are to be construed in an open, inclusive sense; i.e., it is equivalent to “including, but not limited to”.

As used in the specification and claims, the singular for “a”, “an”, and “the” include plural references unless the context clearly dictates otherwise. For example, the term “an excipient” includes a plurality of excipients, including mixtures thereof. Similarly, use of “a compound” for treatment of preparation of medicaments as described herein contemplates using one or more compounds of the disclosure for such treatment or preparation unless the context clearly dictates otherwise

It should be understood that the terms “a” and “an” as used herein refer to “one or more” of the enumerated components. The use of the alternative (e.g., “or”) should be understood to mean either one, both, or any combination thereof of the alternatives.

As used herein, “about” and “approximately” generally refer to an acceptable degree of error for the quantity measured, given the nature or precision of the measurements. Typical, exemplary degrees of error may be within 20%, 10%, or 5% of a given value or range of values. Alternatively, and particularly in biological systems, the terms “about” and “approximately” may mean values that are within an order of magnitude, potentially within 5-fold or 2-fold of a given value. When not explicitly stated, the terms “about” and “approximately” mean equal to a value, or within 20% of that value.

As used herein, numerical quantities are precise to the degree reflected in the number of significant figures reported. For example, a value of 0.1 is understood to mean from 0.05 to 0.14. As another example, the interval of values 0.1 to 0.2 includes the range from 0.05 to 0.24.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

The term “subject” refers to a mammal, such as a domestic pet (for example, a dog or cat), or human. Preferably, the subject is a human.

The phrase “effective amount” refers to the amount which, when administered to a subject or patient for treating a disease, is sufficient to effect such treatment for the disease.

The term “dosage unit form” is the form of a pharmaceutical product, including, but not limited to, the form in which the pharmaceutical product is marketed for use. Examples include, but are not limited to, pills, tablets, capsules, and liquid solutions and suspensions.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, “deficiency” of an enzyme refers to the absence of or reduced levels or activity of the enzyme, or the presence of a defective enzyme having decreased activity or function.

As used herein, “deficiency” of a metabolic product refers to the absence of or reduced levels of a metabolic product.

As used herein, “overexpression” of an enzyme refers to an excess in production or activity of the enzyme.

As used herein, “downstream product” of an enzyme refers to a substance the production of which is dependent upon the activity of the referenced enzyme. Similarly, “downstream product” of a compound refers to a substance the production of which is dependent upon the presence of the referenced compound. For example, acetyl coenzyme A (“Acetyl-CoA”) is a downstream product of Coenzyme A.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol (2-dimethylaminoethanol), 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, caffeine, and meglumine.

The compounds of the invention, or their pharmaceutically acceptable salts, contain one or more asymmetric centers and may thus give rise to enantiomers, diastereoisomers, and other stereoisomeric forms that are defined, in terms of absolute stereochemistry, as (R)- or (S)-, or as (D)- or (L)- for amino acids. The present invention is meant to include all such possible isomers, as well as their racemic, scalemic, and optically pure forms. Optically active (+) and (−), (R)- and (S)-, or (D)- and (L)-, isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The present invention includes all manner of rotamers and conformationally restricted states of a compound of the invention. Atropisomers, which are stereoisomers arising because of hindered rotation about a single bond, where energy differences due to steric strain or other contributors create a barrier to rotation that is high enough to allow for isolation of individual conformers, are also included.

A “stereoisomer” refers to a compound made up of the same atoms bonded by the same bonds but having different three-dimensional structures, which are not superimposable. The present invention contemplates various stereoisomers and mixtures thereof and includes “enantiomers”, which refers to two stereoisomers whose molecules are nonsuperimposeable mirror images of one another. For example, the carbon and phosphorous atoms marked with an “*” in the following structure are stereocenters. All stereoisomers of the compounds disclosed herein are also included in the scope of the invention.

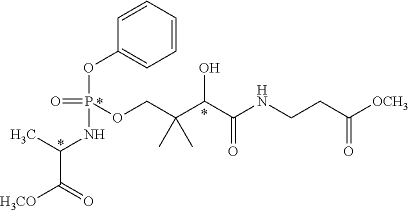 [SMILES: COC(=O)CCNC(=O)C(O)C(C)(C)COP(=O)(NC(C)C(=O)OC)Oc1ccccc1]

The invention disclosed herein is also meant to encompass all pharmaceutically acceptable compounds of the structures disclosed herein being isotopically-labeled by having one or more atoms replaced by an atom of the same element having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of structures disclosed herein, for example, those incorporating a radioactive isotope, are useful in drug and/or substrate tissue distribution studies. These radiolabeled compounds could be useful to help determine or measure the effectiveness of the compounds, by characterizing, for example, the site or mode of action, or binding affinity to a pharmacologically important site of action. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound or formulation of this disclosure to a mammal for a period of time sufficient to yield a metabolic product thereof. Such products are typically identified by administering a radiolabeled compound of the invention in a detectable dose to an animal, such as a rat, mouse, guinea pig, or monkey, or to a human, allowing sufficient time for metabolism to occur, and isolating its conversion products from the urine, blood, or other biological samples.

“Stable compound” and “stable chemical structure” are meant to indicate a compound that is sufficiently robust to survive isolation to a useful degree of purity from a reaction mixture, and formulation into an efficacious therapeutic agent.

When used to describe a pharmaceutical composition, “stability” refers to the maintenance of chemical and physical properties over time. “Chemical stability” refers to the accumulation of degradation products over time. “Physical stability” refers to the maintenance of physical properties, such as hygroscopicity, particle shape, density, flowability, and compactibility.

The term “Hausner ratio” refers to the ratio of tapped density (ρ_{tapped}) to bulk density (ρ_{bulk}) (i.e., Hausner ratio=(ρ_{tapped})/(ρ_{bulk}). Hausner ratios are used to measure flowability of a powder, with lower values indicating better flowability. A generally acceptable scale of flowability expressed in Hausner ratios is provided in The United States Pharmacopeia, 2011, Chapter <1174>. For example, powders having Hausner ratios from 1.00-1.11 have “excellent” flowability; powders having ratios from 1.12-1.18 have “good” flowability; powders having ratios from 1.19 to 1.25 have “fair” flowability; and powders having ratios from 1.26 to 1.34 have “passable” flowability. Powders having Hausner ratios greater of 1.35 or greater are classified as having “poor,” “very poor,” or “very, very poor” flowability. As used herein, tapped densities, bulk densities, and Hausner ratios are determined according to standard procedures set forth in The United States Pharmacopeia, 2011, Chapters <616> and <1174>.

Terms used herein to describe the solubility of a substance are as given in The United State Pharmaceopeia, Chapter <29>, as follows: “very soluble” (less than 1 part solvent for 1 part solute), “freely soluble” (from 1 to 10 parts solvent for 1 part solute), “soluble” (from 10 to 30 parts solvent for 1 part solute), “sparingly soluble” (from 30 to 100 parts solvent for 1 part solute), “slightly soluble” (from 100 to 1000 parts solvent for 1 part solute), “very slightly soluble” (from 1000 to 10,000 parts solvent for 1 part solute), and “practically insoluble, or insoluble” (10,000 or more parts solvent for 1 part solute).

A “pharmaceutical formulation” refers to a formulation of a compound and a medium generally accepted in the art for the delivery of the biologically active compound to mammals, e.g., humans. Such a medium includes all pharmaceutically acceptable carriers, diluents, or excipients therefor, unless otherwise stated.

“Pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Additional definitions are provided throughout the present disclosure.

### Solid Pharmaceutical Formulations and Routes of Administration

In certain aspects, the present invention provides solid pharmaceutical formulations comprising a compound having formula I

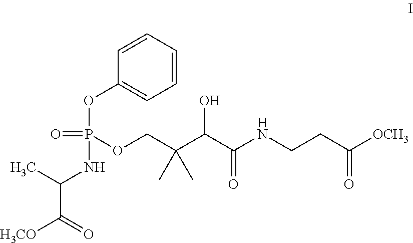 [SMILES: COC(=O)CCNC(=O)C(O)C(C)(C)COP(=O)(NC(C)C(=O)OC)Oc1ccccc1]

or a pharmaceutically acceptable salt thereof. Compound I has the chemical name methyl 3-(2-hydroxy-4-((((1-methoxy-1-oxopropan-2-yl)amino)(phenoxy)phosphoryl)oxy)-3,3-dimethylbutanamido)propanoate. In one embodiment, the pharmaceutical formulation includes an effective amount of the compound of formula I to treat a neurologic disorder.

In one embodiment, a solid pharmaceutical formulation comprising (a) a pharmaceutically acceptable solid excipient, and (b) a compound of formula I or a pharmaceutically acceptable salt thereof, is provided.

In one embodiment, the compound of formula I has the following structure:

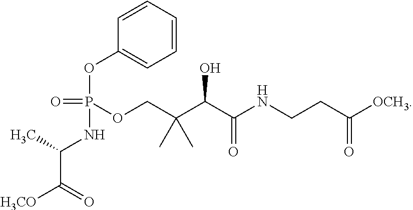 [SMILES: COC(=O)CCNC(=O)[C@H](O)C(C)(C)COP(=O)(N[C@@H](C)C(=O)OC)Oc1ccccc1]

In one embodiment, the excipient has a nominal particle size of greater than or equal to 100 μm. In one embodiment, the nominal particle size of the excipient is greater than or equal to 100 μm. In one embodiment, the nominal particle size of the excipient is greater than or equal to 200 μm. In one embodiment, the nominal particle size of the excipient is greater than or equal to 300 μm.

In one embodiment, the excipient has a pore volume of greater than or equal to 0.01 cm³/g. In one embodiment, the pore volume of the excipient is greater than or equal to 0.1 cm³/g. In one embodiment, the pore volume of the excipient is greater than or equal to 1.0 cm³/g. In one embodiment, the pore volume of the excipient is greater than or equal to 3.0 cm³/g.

In one embodiment, the excipient has a specific surface area greater than or equal to 0.5 m²/g. In one embodiment, the specific surface area of the excipient is greater than or equal to 100 m²/g. In one embodiment, the specific surface area of the excipient is greater than or equal to 200 m²/g. In one embodiment, the specific surface area of the excipient is greater than or equal to 300 m²/g.

In one embodiment, the excipient is selected from the group of microcrystalline cellulose, lactose, calcium hydrogen phosphate, croscarmellose sodium, crosslinked polyvinylpyrrolidine, magnesium stearate, sodium stearyl fumarate, starch 1500, xanthan gum, guar gum, sucralose, gelatin, magnesium aluminometasilicate, hydroxypropyl methylcellulose acetate succinate, hydroxypropyl-β-cyclodextrin, mesoporous silica, and mannitol. In a particular embodiment, the excipient comprises lactose. In a further embodiment, the excipient comprises lactose monohydrate. In another embodiment, the excipient comprises mannitol. In another embodiment, the excipient comprises microcrystalline cellulose.

In one embodiment, the excipient is at least slightly soluble in water at room temperature. In one embodiment, the excipient is at least sparingly soluble in water at room temperature. In one embodiment, the excipient is at least soluble in water at room temperature. In one embodiment, the excipient is at least freely soluble in water at room temperature. In one embodiment, the excipient is at least very soluble in water at room temperature.

In one embodiment, the excipient does not comprise a metal.

In one embodiment, the excipient does not comprise basic compounds.

In any of the aforementioned embodiments, the compound of formula I may comprise from 5% to 80% by weight of the formulation. For example, in particular embodiments, the compound of formula I comprises from 10% to 70% by weight of the formulation; from 20% to 50% by weight of the formulation; from 20% to 30% by weight of said formulation; or from 10% to 20% by weight of the formulation. In a particular embodiment, the compound of formula I comprises 10% to 20% by weight of the formulation. In a further embodiment, the compound of formula I comprises 20% by weight of the formulation.

In any of the aforementioned embodiments, the increase in the amount of total impurities (wt %) after storage at 30° C. and 60% relative humidity for 4 weeks may be less than or equal to 15%. For example, in particular embodiments, the increase is not greater than 15%, not greater than 10%, or not greater than 5%.

In any of the aforementioned embodiments, the formulation may have a Hausner ratio of from 1.0 to 1.50. In a particular embodiment, the formulation has a Hausner ratio of from 1.0 to 1.34. In a further embodiment, the formulation has a Hausner ratio of from 1.0 to 1.25.

The pharmaceutical formulations described herein may be a dosage unit form, such as a tablet, capsule, or sachet. The pharmaceutical formulations of the present invention may be administered by a variety of routes including orally and by injection (e.g., subcutaneously, intravenously, or intraperitoneally). In one embodiment, the pharmaceutical formulation is administered orally in the form of a solid dosage form. For example, the formulation may be provided within a capsule, the contents of which may be mixed with water prior to oral administration. The oral dosage forms may include additional excipients known in the art, such as binders, disintegrating agents, flavorants, antioxidants, and preservatives.

The pharmaceutical formulations disclosed herein may also be administered by injection. Formulations suitable for injection may include sterile powders for the extemporaneous preparation of sterile injectable solutions or dispersions. The formulations may be sterile and be fluid to the extent that easy syringability exists. It may be stable under the conditions of manufacture and storage and be preserved against the contaminating action of microorganisms such as bacteria and fungi. The carrier can be a solvent or dispersion medium containing, for example, water, ethanol, polyol (such as, glycerol, propylene glycol, and liquid polyethylene glycol), suitable mixtures thereof, and vegetable oils. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, and ascorbic acid. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate, or gelatin.

Sterile injectable solutions can be prepared by incorporating the formulations containing the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

### Uses and Methods of Treatment

An additional aspect of the present invention is a formulation as disclosed herein for use in treating a neurologic disorder.

Also provided is a formulation according to the present disclosure for use in treating a disorder associated with pantothenate kinase enzyme deficiency. In one embodiment, the disorder is pantothenate kinase associated neurodegeneration. In another embodiment, the disorder is 4′ phosphopantothenic acid deficiency. In yet another embodiment, the subject exhibits neurodegeneration with brain iron accumulation. In a further embodiment, the subject has a pantothenate kinase gene (PANK) defect, such as a PANK1 gene defect, a PANK2 gene defect, a PANK3 gene defect, a PANK4 gene defect, or any combination thereof.

Also provided is a formulation according to the present disclosure for use in treating a subject having a disorder associated with Coenzyme A deficiency.

Yet another aspect of the present disclosure is a formulation as disclosed herein for use in treating a condition associated with abnormal neuronal function in a subject in need thereof. In one embodiment, the condition is Parkinson's disease, dystonia, extrapyramidal effects, dysphagia, rigidity and/or stiffness of limbs, choreoathetosis, tremor, dementia, spasticity, muscle weakness, or seizure.

Yet another aspect of the present disclosure is a formulation as disclosed herein for use in treating a condition associated with neuronal cell iron accumulation in a subject in need thereof.

Yet another aspect of the present disclosure is a formulation as disclosed herein for use in treating a subject having neurodegeneration with brain iron accumulation.

In any of the above embodiments, the formulation for use may be mixed with water for administration.

Yet another aspect is a method of increasing Coenzyme A production or 4′-phosphopantothenic acid production in a subject in need thereof by administering to the subject an effective amount of a formulation of the present invention. In one embodiment, the subject in need of increased Coenzyme A production or 4′ phosphopantothenic acid production exhibits overexpression of an enzyme for which Coenzyme A is a substrate or synthetic precursor. In one embodiment, the subject in need of increased Coenzyme A production or 4′ phosphopantothenic acid production has a deficiency of Coenzyme A production, a deficiency of pantothenate kinase enzyme, and/or a deficiency of 4′-phosphopantothenic acid. In one embodiment, the subject in need thereof has a defect or mutation in a pantothenate kinase gene (PANK). In one embodiment, a method of increasing Coenzyme A production or 4′ phosphopantothenic acid production in a subject having a defect in the PANK1, PANK2, PANK3, or PANK4 gene, or any combination thereof, is provided. In one embodiment, a method of increasing Coenzyme A production or 4′ phosphopantothenic acid production in a subject having a defect in the PANK2 gene is provided.

Yet another embodiment is a method of treating a subject having a disorder associated with pantothenate kinase enzyme deficiency comprising administering to a subject in need thereof an effective amount of a formulation according to the present invention. In one embodiment, the disorder is pantothenate kinase-associated neurodegeneration (PKAN). In one embodiment, the disorder is 4′-phosphopantothenic acid deficiency. In another embodiment, the subject exhibits neurodegeneration with brain iron accumulation. In one embodiment, the subject having a disorder associated with pantothenate kinase enzyme deficiency has a pantothenate kinase gene (PANK) defect. In one embodiment, a method of treating a subject having a disorder associated with pantothenate kinase enzyme deficiency, PKAN, 4′ phosphopantothenic acid deficiency, or neurodegeneration with brain iron accumulation is provided, wherein the subject has a defect in the PANK1, PANK2, PANK3, or PANK4 gene, or any combination thereof. In one embodiment, a method of treating a subject having a disorder associated with pantothenate kinase enzyme deficiency is provided, wherein the subject has a PANK1 gene defect. In one embodiment, a method of treating a subject having a disorder associated with pantothenate kinase enzyme deficiency is provided, wherein the subject has a PANK2 gene defect. In one embodiment, a method of treating a subject having a disorder associated with pantothenate kinase enzyme deficiency is provided, wherein the subject has a PANK3 gene defect. In one embodiment, a method of treating a subject having a disorder associated with pantothenate kinase enzyme deficiency is provided, wherein the subject has a PANK4 gene defect.

Yet another embodiment is a method of treating a subject having a disorder associated with Coenzyme A deficiency, comprising administering to the subject an effective amount of a formulation of the present invention.

Yet another embodiment is a method of treating a condition associated with abnormal neuronal function in a subject, comprising administering to the subject an effective amount of a formulation of the present invention. In one embodiment, the condition is Parkinson's disease, dystonia, extrapyramidal effects, dysphagia, rigidity and/or stiffness of limbs, choreoathetosis, tremor, dementia, spasticity, muscle weakness, or seizure.

Yet another embodiment is a method of treating a condition associated with neuronal cell iron accumulation in a subject in need thereof, comprising administering to the subject an effective amount of a formulation of the present invention.

Another embodiment is a method of treating a subject having neurodegeneration with brain iron accumulation, comprising administering to the subject an effective amount of a formulation of the present invention. In one embodiment, the subject having neurodegeneration with brain iron accumulation has pantothenate kinase-associated neurodegeneration (PKAN).

In any of the aforementioned embodiments, the subject being treated or in need thereof may be a child. In one embodiment, the child is 10 to 15 years old. In another embodiment, the subject being treated or in need thereof is an adult.

In addition to being used as a monotherapy, the formulations disclosed herein may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

The additional agent or agents may be selected from any agent or agents useful for treating a neurological disorder, for example any agent or agents useful for treating a deficiency of pantothenate kinase, 4′-phosphopantothenic acid, or Coenzyme A. In one embodiment, the additional agent or agents is useful in improving cognitive function. For example, the additional agent or agents may be an acetylcholinesterase inhibitor, such as physostigmine, neostigmine, pyridostigmine, ambenonium, demarcarium, rivastigmine, galantamine, donezepil, and combinations thereof. In another embodiment, the additional agent or agents is an iron chelator, such as deferiprone, deferoxamine, deferasirox, and combinations thereof.

The actual dosage amount of the compound of formula I, or pharmaceutical salt thereof, administered to a subject may be determined by physical and physiological factors such as age, sex, body weight, severity of condition, the type of disease being treated, previous or concurrent therapeutic interventions, idiopathy of the subject, and the route of administration. These factors may be determined by a skilled artisan. The practitioner responsible for administration will typically determine the concentration of active ingredient(s) in a composition and appropriate dose(s) for the individual subject.

In one embodiment, a human subject is administered a daily dose of a compound having formula I from about 0.01 mg/kg to about 100 mg/kg.

In one embodiment, the dose of a compound having formula I administered to a human subject is 300 mg. In one embodiment, the dose of a compound having formula I administered to a human subject is 150 mg. In one embodiment, the dose of a compound having formula I administered to a human subject is 100 mg. In one embodiment, the dose of a compound having formula I administered to a human subject is 75 mg. In one embodiment, the dose of a compound having formula I administered to a human subject is 50 mg.

Single or multiple doses of the formulations are contemplated. Desired time intervals for delivery of multiple doses can be determined by one of ordinary skill in the art employing no more than routine experimentation. As an example, subjects may be administered two doses daily at approximately 12 hour intervals. In one embodiment, the formulation is administered once a day. In another embodiment, the formulation is administered two times a day. In another embodiment, the formulation is administered three times a day.

In one embodiment, the aforementioned methods comprise administering a 300 mg dose of fosmetpantotenate to a human subject three times a day. In one embodiment, the aforementioned methods comprise administering fosmetpantotenate at a dose from 50 to 150 mg to a human subject three times a day. In one embodiment, the aforementioned methods comprise administering a 50 mg dose of fosmetpantotenate to a human subject three times a day. In one embodiment, the aforementioned methods comprise administering a 75 mg dose of fosmetpantotenate to a human subject three times a day. In one embodiment, the aforementioned methods comprise administering a 100 mg dose of fosmetpantotenate to a human subject three times a day. In one embodiment, the aforementioned methods comprise administering a 150 mg dose of fosmetpantotenate to a human subject three times a day.

The formulations may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis, or any set number of days or weeks there-between. Alternatively, the predetermined routine schedule may involve administration on a twice daily basis for the first week, followed by a daily basis for several months. In other embodiments, the invention provides that the agent(s) may be taken orally and that the timing of which is or is not dependent upon food intake. Thus, for example, the agent can be taken every morning and/or every evening, regardless of when the subject has eaten or will eat.

In one embodiment, any of the aforementioned methods may comprise mixing the formulation with water and administering the resulting mixture.

### EXAMPLES

### Example 1

### Synthesis of Fosmetpantotenate

Synthesis of Fosmetpantotenate

Fosmetpantotenate was synthesized as follows.

### Step 1: Manufacture of Intermediate 1 (Methyl Pantothenate)

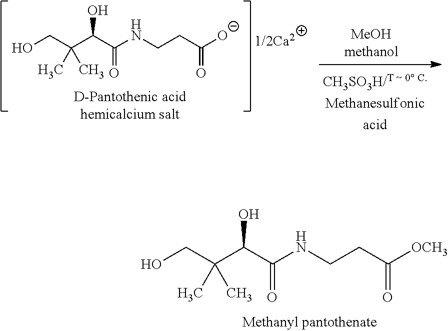

D-pantothenic acid hemicalcium salt (D-PAHS) was charged to a reactor, followed by the addition of methanol. The solution was mixed and cooled (−5˜0° C.) to effect dissolution. Methanesulfonic acid was slowly added (over a period of 1 hour) to the D-PAHS solution, while maintaining a temperature at −5˜0° C. The reaction mixture was allowed to stir at −5˜0° C. for not less than 10 hours. The reaction was complete when the D-PAHS content was not more than 1.0%.

### Step 2: Manufacture of Intermediate 2 (Crude Fosmetpantotenate)

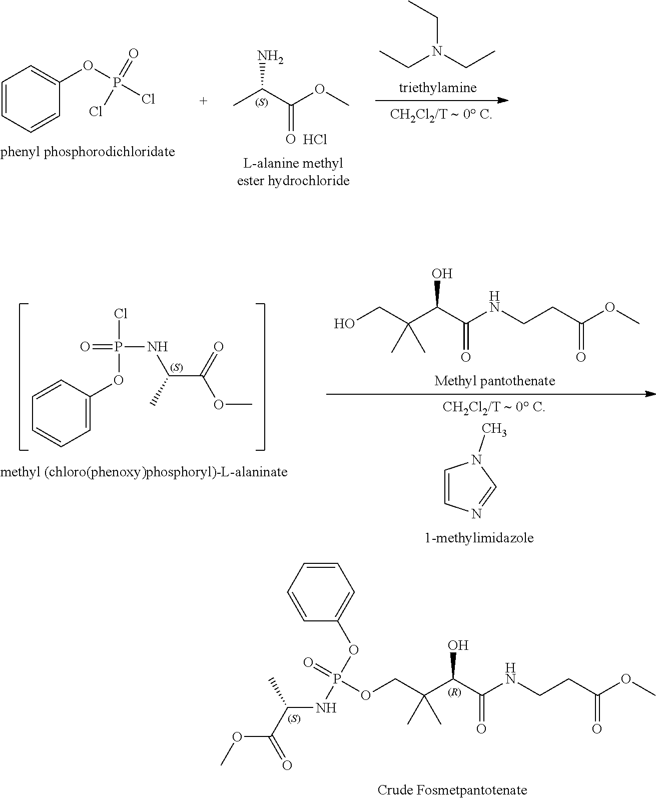

An appropriate sized reactor was purged with and maintained under nitrogen throughout the manufacturing process. L-alanine methyl ester hydrochloride was charged to the reactor, followed by the addition of dichloromethane (DCM), and allowed to mix at −60˜−50° C. to effect dissolution. Over a period of 0.5 hours, phenyl dichlorophosphate was slowly added, while maintaining a temperature at −60˜−50° C. A solution containing triethylamine in DCM was charged to the reactor over a period of 2-4 hours while maintaining a temperature at −60˜−50° C. The reaction mixture was stirred at −60˜−50° C. for 5-10 hours. The reaction was complete when the Phenyl dichlorophosphate content was not more than 20%.

In an alternative synthesis, a mixture of CH₂Cl₂ and 2-methyl-tetrahydrofurane provided at −50° C. was used in place of CH₂Cl₂ at ˜0° C. (to reduce side reactions).

### Step 3: Chromatographic Purification of Fosmetpantotenate

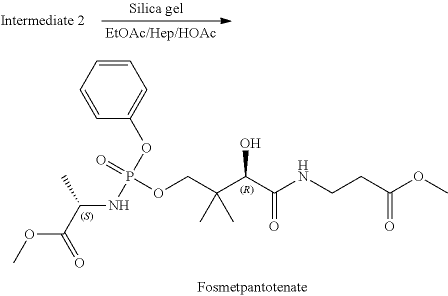

Chromatographic purification of the crude fosmetpantotenate (“Intermediate 2”) was achieved by packing an appropriately sized chromatography column with silica gel. The crude compound, was loaded onto the column and eluted isocratically with a mobile phase consisting of 50% ethyl acetate, 50% n-heptane (Hep), and 0.1% acetic acid (HOAc) (w/w/w).

### Characterization of Purified Fosmetpantotenate

Purified fosmetpantotenate is a viscous liquid. Table 1 shows the viscosity of compositions comprising fosmetpantotenate and various liquid excipients or solvents.

TABLE 1
Viscosity (Cp) for compositions comprising fosmetpantotenate
(“F”) and various liquid excipients or solvents, as well as
viscosity of reference substances.

| Excipient | Viscosity (Cp) |
|---|---|
| 6.3% acetic acid + F | 15000 |
| 9.0% acetic acid + F | 9000 |
| 18.0% acetic acid + F | 1700 |
| 38% LG-90 (Lauroglycol 90, propylene | 580 |
| glycol monolaurate (type II)) + F | |
| 36% Capmul® MCM + F | 3440 |
| 24% propylene glycol + F | 1000 |
| 15% caprylic acid + F | 2750 |
| 8% ethyl acetate + F | 8700 |
| 9% ethyl lactate + F | 7000 |
| 24% triethyl citrate + F | 6500 |
| 26% dimethyl isosorbide (abrasolve) + F | 5000 |

Reference substances

| | |
|---|---|
| Water | 1 |
| Karo syrup | 5000 |
| Honey | 10,000 |

### Example 2

### Microcrystalline Cellulose Formulation

Fosmetpantotenate (0.6 g) was first mixed with ethyl acetate at a 10:1 weight/weight ratio to form a solution, then blended with microcrystalline cellulose (“MCC,” Avicel® PH-200) using a mortar and pestle by adding the solution in portions and mixing to achieve 20% w/w fosmetpantotenate. The powder was tray dried overnight at ambient temperature.

### Example 3

### Mannitol Formulation

Fosmetpantotenate (0.6 g) was first mixed with ethyl acetate at a 10:1 weight/weight ratio to form a solution, then blended with mannitol (Parteck® M 200) using a mortar and pestle by adding the solution in portions and mixing to achieve 20% w/w fosmetpantotenate. The powder was tray dried overnight at ambient temperature.

### Example 4

### Lactose Monohydrate Formulation

Fosmetpantotenate (0.6 g) was first mixed with ethyl acetate at a 10:1 weight/weight ratio to form a solution, then blended with lactose monohydrate (316 Fast Flo®) using a mortar and pestle by adding the solution in portions and mixing to achieve 20% w/w fosmetpantotenate. The powder was tray dried overnight at ambient temperature.

### Example 5

### Mesoporous Silica Formulation

Fosmetpantotenate (5 g) was mixed with ethyl acetate (380 g) to form a solution. Mesoporous silica (Parteck® SLC, ˜20 μm particle size, 15 g) was added into the solution to achieve a total of 5% solids by weight. The resulting suspension was spray-dried using (Büchi B-290) at a 10 g/min spray rate, 26 psi atomization pressure, 110° C. inlet temperature, and 70° C. outlet temperature. The resulting powder was tray-dried for 24 hours at 35° C.

### Example 6

### Microcrystalline Cellulose Formulation

Fosmetpantotenate (287.7 g) and 287.7 g ethyl acetate were mixed in a stainless kettle using an overhead stirrer (150 rpm, 17 min). The solution was sprayed onto 1000 g of microcrystalline cellulose (Avicel® PH-200) in a PMA 10 granulator (impeller speed set to 400 rpm) using a peristaltic pump at the rate of 20 g/min. An additional 25 mL ethyl acetate was used to rinse the kettle and tubing. The granulator was run for an additional 15 minutes, before unloading the powder onto drying trays and drying at ambient temperature for 1 hour.

### Example 7

### Microcrystalline Cellulose Formulations with Fosmetpantotenate Loadings of 20-45%

Microcrystalline cellulose (Avicel® PH-200) was sieved via No. 120 mesh to remove fines prior to drug loading experiments. A solution of 50 wt % fosmetpantotenate in ethyl acetate was added dropwise using a peristaltic pump at the rate of 4 g/min to 80 g of the sieved microcrystalline cellulose in a high shear granulator (GMX Lab Micro High Shear Granulator), at ambient temperature. The impeller speed was set to 100 rpm (chopper was not used). Drug loading was increased in 5% increments from 20%, by adding the desired amount of fosmetpantotenate solution into the powder bed each time followed by 2 hours of additional mixing for drying purposes, with the impeller speed set to 125 rpm, until failure (lumpiness, loss of powder flow, etc.) was observed. Sample powders of different drug loadings were taken throughout the process and evaluated for appearance and flowability.

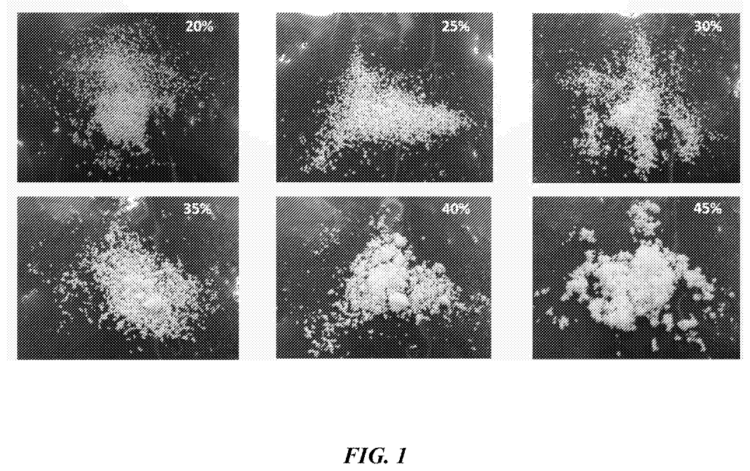

As shown in FIG. 1, at drug loadings 20%, 25%, and 30%, the resulting solid formulation was a free-flowing powder with no obvious appearance change compared to the microcrystalline cellulose. At 35% drug loading, the formulation was a free-flowing powder but contained a small amount of loose agglomerates. At 40% drug loading, the formulation was a flowable powder with an increased amount of loose agglomerates relative to the 35% drug loading formulation. Finally, the 45% drug loading formulation exhibited poor flowability; materials appeared to be wet and showed large amounts of loose agglomerates.

### Example 8

### Dibasic Calcium Phosphate Formulation

Fosmetpantotenate (0.6 g) was first mixed with ethyl acetate at a 10:1 weight/weight ratio to form a solution, then blended with dibasic calcium phosphate) (Emcompress®) using a mortar and pestle by adding the solution in portions and mixing to achieve 20% w/w fosmetpantotenate. The powder was tray dried overnight at ambient temperature.

### Example 9

### Croscarmellose Sodium Formulation

Fosmetpantotenate (0.6 g) was first mixed with ethyl acetate at a 10:1 weight/weight ratio to form a solution, then blended with croscarmellose sodium (Ac-Di-Sol® SD-711) using a mortar and pestle by adding the solution in portions and mixing to achieve 20% w/w fosmetpantotenate loading. The powder was tray dried overnight at ambient temperature.

### Example 10

### Crosslinked Polyvinylpyrrolidone Formulation

Fosmetpantotenate (0.6 g) was first mixed with ethyl acetate at a 10:1 weight/weight ratio to form a solution, then blended with crosslinked polyvinylpyrrolidone (Kollidon® CL) using a mortar and pestle by adding the solution in portions and mixing to achieve 20% w/w fosmetpantotenate. The powder was tray dried overnight at ambient temperature.

### Example 11

### Magnesium Stearate Formulation

Fosmetpantotenate (0.6 g) was first mixed with ethyl acetate at a 10:1 weight/weight ratio to form a solution, then blended with magnesium stearate using mortar and pestle by adding the solution in portions and mixing to achieve 20% w/w fosmetpantotenate. The powder was tray dried overnight at ambient temperature.

### Example 12

### Sodium Stearyl Fumarate Formulation

Fosmetpantotenate (0.6 g) was first mixed with ethyl acetate at a 10:1 weight/weight ratio to form a solution, then blended with sodium stearyl fumarate using a mortar and pestle by adding the solution in portions and mixing to achieve 20% w/w fosmetpantotenate. The powder was tray dried overnight at ambient temperature.

### Example 13

### Starch Formulation

Fosmetpantotenate (0.6 g) was first mixed with ethyl acetate at a 10:1 weight/weight ratio to form a solution, then blended with starch (Starch 1500) using a mortar and pestle by adding the solution in portions and mixing to achieve 20% w/w fosmetpantotenate. The powder was tray dried overnight at ambient temperature.

### Example 14

### Xanthan Gum Formulation

Fosmetpantotenate (0.6 g) was first mixed with ethyl acetate at a 10:1 weight/weight ratio to form a solution, then blended with xanthan gum using a mortar and pestle by adding the solution in portions and mixing to achieve 20% w/w fosmetpantotenate. The powder was tray dried overnight at ambient temperature.

### Example 15

### Guar Gum Formulation

Fosmetpantotenate (0.6 g) was first mixed with ethyl acetate at a 10:1 weight/weight ratio to form a solution, then blended with guar gum using a mortar and pestle by adding the solution in portions and mixing to achieve 20% w/w fosmetpantotenate. The powder was tray dried overnight at ambient temperature.

### Example 16

### Sucralose Formulation

Fosmetpantotenate (0.6 g) was first mixed with ethyl acetate at a 10:1 weight/weight ratio to form a solution, then blended with sucralose using a mortar and pestle by adding the solution in portions and mixing to achieve 20% w/w fosmetpantotenate. The powder was tray dried overnight at ambient temperature.

### Example 17

### Magnesium Aluminometasilicate Formulation

Fosmetpantotenate (0.6 g) was first mixed with ethyl acetate at a 10:1 weight/weight ratio to form a solution, then blended with magnesium aluminometasilicate (Neusilin® US2) using a mortar and pestle by adding the solution in portions and mixing to achieve 20% w/w fosmetpantotenate. The powder was tray dried overnight at ambient temperature.

### Example 18

### Gelatin Formulation

Fosmetpantotenate (0.6 g) was first mixed with ethyl acetate at a 10:1 weight/weight ratio to form a solution, then blended with gelatin (NF Gelatin Powder) using a mortar and pestle by adding the solution in portions and mixing to achieve 20% w/w fosmetpantotenate. The powder was tray dried overnight at ambient temperature.

### Example 19

### Hydroxypropyl Methylcellulose Acetate Succinate Formulation

Fosmetpantotenate (0.6 g) was first mixed with ethyl acetate at a 10:1 weight/weight ratio to form a solution, then blended with hydroxypropyl methylcellulose acetate succinate (HPMCAS-L; also referred to as hypromellose acetate succinate) using a mortar and pestle by adding the solution in portions and mixing to achieve 20% w/w fosmetpantotenate. The powder was tray dried overnight at ambient temperature.

### Example 20

### Hydroxypropyl Beta Cyclodextrin Formulation

Fosmetpantotenate (0.6 g) was first mixed with ethyl acetate at a 10:1 weight/weight ratio to form a solution, then blended with hydroxypropyl beta cyclodextrin using a mortar and pestle by adding the solution in portions and mixing to achieve 20% w/w fosmetpantotenate. The powder was tray dried overnight at ambient temperature.

### Example 21

### Mesoporous Silica Formulation

Fosmetpantotenate (0.6 g) was first mixed with ethyl acetate at a 10:1 weight/weight ratio to form a solution, then blended with mesoporous silica (Parteck® SLC) using a mortar and pestle by adding the solution in portions and mixing to achieve 20% w/w fosmetpantotenate. The powder was tray dried overnight at ambient temperature.

### Example 22

### Maximum Drug Loading for Highly Porous Substrates

Fosmetpantotenate was first thinned with ethyl acetate at a 10:1 weight/weight ratio, followed by being loaded onto inorganic and organic porous substrates via a mortar and pestle. The maximum fosmetpantotenate-to-powder loading ratio was determined by failure mode (e.g., liquidification, lumpiness, loss of powder flow). At 1:4 API:substrate, 1 g product carries 200 mg fosmetpantotenate. For formulations comprising mesoporous silica or magnesium aluminometasilicate as the porous substrate, the highest loading is approximately 66% and 50%, respectively, as shown below in Table 2.

TABLE 2
Loading of fosmetpantotenate onto highly porous substrates.

| | | | | | Maximum |
|---|---|---|---|---|---|
| | | | | | fosmet- |
| | | | | | pantotenate |
| | | | | | loading |
| | | | | Specific | capacity |
| | Brand | Nominal | Pore | surface | (w/w fosmet- |
| | name & | particle | volume | area | pantotenate/ |
| Substrate | grade | size (μm) | (cm³/g) | (m²/g) | substrate) |
| mesoporous | Syloid® | 150 | 2.2 | 320 | 2:1 |
| silica | XDP | | | | |
| magnesium | Neusilin® | 100 | 0.9 | 300 | 1:1 |
| aluminometa- | US2 | | | | |
| silicate | | | | | |

### Example 23

### Stability of Solid Fosmetpantotenate Formulations

Formulations with 10% drug loading were made using the solid excipients shown in Table 3. Fosmetpantotenate was thinned with ethyl acetate (EtOAc) at a 1:1 weight/weight ratio, and then blended with each solid excipient using a mortar and pestle on the 0.1 g scale of fosmetpantotenate, i.e., 0.2 g 50% (fosmetpantotenate/EtOAc)+0.9 g solid. The resulting materials were dried overnight at room temperature in a HEPA hood to remove excess EtOAc.

The formulations were stored under accelerated stability conditions (25° C./60% relative humidity (RH)) for 4 weeks. Some samples were stored in “open” conditions, i.e., exposed to air; other samples were stored in closed containers. The amount of impurities was measured at beginning of storage (t=0), after 2 weeks of storage (t=2 weeks), and after 4 weeks of storage (t=4 weeks). The amount of impurities (total related substances, expressed as a percentage) was determined using the methods described in Example 26 below.

TABLE 3
Accelerated stability data for 10% drug loaded formulations.

| | Total % Related Substances | | | | |
|---|---|---|---|---|---|
| | | t = 2 weeks | | t = 4 weeks | |
| Excipient | t = 0 | Open | Closed | Open | Closed |
| None | 4.80 | 5.94 | 5.76 | 6.29 | 6.39 |
| (fosmetpantotenate) | | | | | |
| Avicel® PH-200 (MCC) | 4.61 | 4.92 | 5.04 | 5.15 | 5.49 |
| Parteck® M 200 (mannitol) | 4.76 | 5.13 | 5.19 | 6.04 | 5.51 |
| Syloid® XDP (silica) | 4.81 | 6.01 | 6.06 | 6.56 | 5.97 |
| Neusilin® US2 | 4.58 | 6.80 | 6.38 | 15.59 | 13.90 |
| (MgO—Al₂O₃—SiO₂) | | | | | |
| 316 Fast Flo® (lactose) | 4.61 | 5.12 | 5.19 | 5.30 | 5.47 |
| Sodium stearyl fumarate | 4.59 | 5.81 | 5.87 | 7.08 | 7.81 |
| Starch 1500 | 4.81 | 6.02 | 5.96 | 6.10 | 6.50 |
| HPMCAS-L | 5.09 | 6.12 | 6.28 | 6.64 | 6.74 |
| HP-β-Cyclodextrin | 5.02 | 6/24 | 6.38 | 6.63 | 7.03 |

### Example 24

### Spray-Dried Hydroxypropyl Methylcellulose Acetate Succinate Formulation

Fosmetpantotenate was dispersed in ethyl acetate and mixed with hydroxypropyl methylcellulose acetate succinate (HPMCAS-L), resulting in a spray drying solution of 5% W/W (HPMCAS-L+fosmetpantotenate). The resulting solution was spray-dried using (Büchi B-290) at a 15-18 g/min spray rate, 25-28 psi atomization pressure, 115-120° C. inlet temperature, and 65-70° C. outlet temperature. The resulting powder was tray-dried for 15 hours at 40° C. in a convection oven.

The resulting particles exhibited poor flowability of SDD due to small particle size (˜10 μm). Therefore, a simulated dry granulation process (“slug and mill”) was conducted to generate free flowing granular powders using a single punch press. The components shown in Table 4 were combined to result in the final formulation, HPMCAS-L SDD, having 12.5% drug loading.

TABLE 4
Components used in the HPMCAS-L SDD formulation.

| Component/Grade | % w/w | Target Weight (g) |
|---|---|---|
| 25:75 RE-024:HPMCAS-L | 50.00 | 20.00 |
| SDI/NA | | |
| Avicel® PH-200 MCC/USP | 21.00 | 8.40 |
| Sodium Parteck® M 200 | 24.75 | 9.90 |
| Mannitol/USP | | |
| Kollidon® CL/USP | 4.00 | 1.60 |
| Magnesium Stearate 2257/NF | 0.25 | 0.10 |
| Total | 100.00 | 40.00 |

### Example 25

### Flowability of Solid Formulations

Solid formulations of fosmetpantotenate were prepared and analyzed for flowability. A solid microcrystalline cellulose formulation and a solid mannitol formulation, each with 20% drug loading, were prepared as in Examples 2 and 3, respectively. A spray-dried hydroxypropyl methylcellulose acetate succinate formulation was prepared as in Example 24. Bulk density and tapped density were determined using standard methods set forth in The United States Pharmacopeia, 2011, Chapters <616> and <1174>.

Table 5 shows the Hausner ratio calculated for each of the formulations, and for samples of mannitol (Parteck® M 200) and microcrystalline cellulose (Avicel® PH-200) as provided by the manufacturer without the addition of any active agent. The Hausner ratios for the solid mannitol and solid microcrystalline cellulose formulations indicate fair flowability.

TABLE 5
Flowability for solid fosmetpantotenate formulations.

| | Bulk density | Tapped density | |
|---|---|---|---|
| Sample | (g/mL) | (g/mL) | Hausner ratio |
| Mannitol (Parteck® | 0.42 | 0.52 | 1.24 |
| M 200) | | | |
| 20% drug mannitol | 0.54 | 0.66 | 1.23 |
| (Parteck® M 200) | | | |
| formulation | | | |
| Microcrystalline | 0.32 | 0.40 | 1.22 |
| cellulose (Avicel® | | | |
| PH-200) | | | |
| 20% drug | 0.41 | 0.50 | 1.24 |
| microcrystalline | | | |
| cellulose (Avicel® | | | |
| PH-200) formulation | | | |
| 12.5% drug | 0.41 | 0.58 | 1.43 |
| HPMCAS-L SDD | | | |
| granulation | | | |

### Example 26

### Chemical Stability of Solid Formulations

A stability study was performed to assess the stability of fosmetpantotenate in the following formulations: 1 g fosmetpantotenate (“drug only”; containing approximately 10% acetic acid); 75 mg fosmetpantotenate EA capsules (10% ethyl acetate); 75 mg fosmetpantotenate LG-90 capsules (38% laurylglycol 90); fosmetpantotenate MCC granulation, as described in Example 2; and fosmetpantotenate mannitol granulation, as described in Example 3; and 25:75 fosmetpantotenate:HPMCAS-L Spray Dried Dispersion (SDD) granulation, as described in Example 24. Additionally, fosmetpantotenate without solid excipient or solvent (“drug only”) was stored closed in borosilicate glass vials. Formulations contained in soft gel capsules were stored in closed HDPE bottles, and solid fosmetpantotentate granulation formulations were stored in closed foil-foil packaging. Stability samples were stored at ca. −20° C. (control samples) and at 30° C. and 65% relative humidity in closed containers for 12 weeks.

The amount of total impurities was determined via integration of HPLC peak areas within 3 days after the formulations were prepared and before beginning storage (time=0 weeks). The amount of total impurities was measured again after 2 weeks, 4 weeks, 6 weeks, 8 weeks, and 12 weeks of storage. Capsule samples for analysis were prepared by dissolving 5 capsules in a volumetric flask with H₂O/ACN 50/50 (v/v) containing 0.1% (v/v) HCOOH. “Drug only” and granulation samples were prepared by adding the sample directly into a volumetric flask and diluting with H₂O/ACN 50/50. The HPLC method used to analyze the samples is shown in Tables 6 and 7.

TABLE 6
HPLC method used to determine amount of impurities.

| Parameter | Value |
|---|---|
| Column | XBridge C18 BEH, 150 × 4.6 mm |
| | with 2.5 μm particles |
| Mobile Phase A | H₂O containing 0.05% (v/v) |
| | HCOOH |
| Mobile Phase B | ACN containing 0.05% (v/v) |
| | HCOOH |
| Program Type | Gradient - see below |
| Flow Rate | 0.8 mL/min |
| Column Temperature | 30° C. |
| Sample Temperature | Room Temperature |
| Injection Volume | 15 μL |
| Needle Wash | H₂O/ACN 50/50 (v/v) containing |
| | 0.1% (v/v) HCOOH |
| Detection Method | UV |
| Detection Wavelength | 210 nm |
| Detection Bandwidth | 4 nm |
| Slit Width | 4 nm |
| Reference Wavelength | off |
| Reference Bandwidth | n/a |
| Run Time | 50 min |
| Fosmetpantotenate ~retention time | Two isomers at 25.0 and 25.5 min |

TABLE 7
HPLC method used to determine amount of impurities.

| Time | | |
|---|---|---|
| (min) | % Mobile Phase A | % Mobile Phase B |
| 0 | 98 | 2 |
| 30 | 50 | 50 |
| 40 | 50 | 50 |
| 40.1 | 98 | 2 |
| 50 | 98 | 2 |

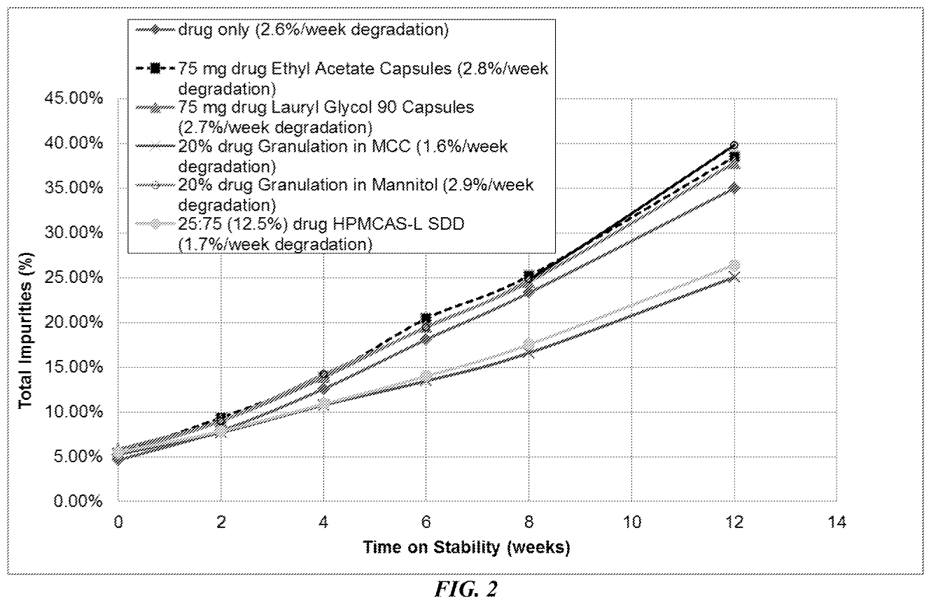

FIG. 2 shows the amount of total impurities (expressed as a percentage) for each of the formulations before storage and after 2, 4, 6, 8, and 12 weeks of storage at ca. −20° C. (control samples) and at 30° C. and 65% relative humidity in closed containers. The average amount of degradation per week was 2.6% for fosmetpantotenate without solid excipient (labeled “dug only” in FIG. 2). The relative amount of total impurities for the solid formulations was similar or less than that for the drug only formulation or the drug formulated with ethyl acetate or lauryl glycol 90, at each time point. Two formulations, the 20% drug loaded microcrystalline cellulose formulation and the 12.5% HPMCAS-L SDD granulation formulation, had lower percent total impurities when measured after 12 weeks of storage.

### Example 27

### Half-Life of Coenzyme A

The half-life of Coenzyme A (CoA) was determined for both wild-type and PanK2-deficient IRM32 cells.

Stable isotopically labeled pantothenic acid (vitamin B5) and fosmetpantotenate, a compound currently in a Phase 3 clinical trial for the treatment of pantothenate associated neurodegeneration, were prepared. The PanK2-deficient cell line was treated with labeled fosmetpantotenate (100 μM) while the parental IRM32 (WT) line was treated with labeled pantothenic acid. After 24 h the media was changed to complete media and the cells were harvested at various time points. The fraction of labeled CoA, both free and total, with respect to time 0 was calculated to determine the half-life of CoA under each of these conditions.

The half-life of CoA was determined to be ˜20 to 48 h depending on the conditions. The half-life of CoA appears to be somewhat shorter in PanK2-deficient cells than in WT cells. It is known that the Pank enzymes are subject to feedback inhibition by CoA, but this regulatory mechanism should be reduced with deficient Pank activity. These results indicate that there may be little to no regulation of the degradation pathways of CoA.

### Example 28

### Alternative Method of Chromatographic Purification of Crude Fosmetpantotenate

Crude fosmetpantotenate was synthesized according to Steps 1 and 2 of Example 1 above. Then, beginning with crude fosmetpantotenate (“Intermediate 2” in Step 3 of Example 1), chromatographic purification was achieved by packing an appropriately sized chromatography column with silica gel, loading the crude compound onto the column, and eluting isocratically with a mobile phase consisting of 25% isopropyl alcohol and 75% n-heptane. After removal of the solvents under reduced pressure, purified fosmetpantotenate that does not contain acetic acid was obtained.

### Example 29

### Alternative Microcrystalline Cellulose Formulation

Fosmetpantotenate (3286.0 g) was prepared using the “no acid” purification process of Example 28 and mixed with 3290.0 g ethyl acetate in a stainless kettle using an overhead stirrer (150 rpm, 17 min). The solution was sprayed onto 10714 g of microcrystalline cellulose (MCC, Avicel® PH-200) in a PMA 65 High Shear Granulator (impeller speed set to 200 rpm) using a peristaltic pump over 27 min. An additional small amount of ethyl acetate was used to rinse the kettle and tubing and sprayed. The resulting granulation was transferred to a Glatt GPCG-30 Fluid Bed granulator and dried at 25° C. (560 m³/hr process air flow) for 29 min. 13224 g of the granulation was obtained at the end of the drying step.

The batch was combined with another batch (13379 g) of granulation prepared in the same manner, an additional 8159 g of microcrystalline cellulose (Avicel® PH-200), and 709 g of Colloidal Silicon Dioxide M5-P. All components were blended in a Bohle Drum Blender for 10 min at 6 rpm to produce 35092 g of the final blend.

### Example 30

### Flowability and Chemical Stability of Solid Formulations Prepared without Acetic Acid

Fosmetpantotenate was prepared and purified as in Examples 1 (“with acetic acid”) and Example 28 (“without acid”). Flowability and stability were analyzed for the acetic acid-containing fosmetpantotenate and the “without acid” fosmetpantotenate, alone and when formulated with MCC.

For the MCC formulations, fosmetpantotenate was diluted 1:1 (w:w) with ethyl acetate (HPLC grade, EMD) to generate a solution of viscosity suitable for wet granulation. Porous MCC (Avicel® PH200) was sieved via 120 mesh (125 μm opening) to remove fine particles and then placed in a Freund-Vector GMX-LAB Micro high shear granulator (1 L stainless steel bowl). The impeller speed was kept at 125 rpm during the process. 40 g of 50 wt % fosmetpantotenate ethyl acetate solution (contains 20 g fosmetpantotenate) was added into 80 g substrate at approximately 8 g/min drop-wise via a glass transfer pipette to achieve a 20:80 fosmetpantotenate:MCC ratio (i.e., 20% drug loading by weight). The resulting materials were agitated for an additional 10 minutes followed by tray drying overnight at room temperature to facilitate removal of ethyl acetate.

### Flowability

Several methods for measuring powder flow through an orifice are discussed in USP chapter <1174> Powder Flow. These methods measure powder flow under the influence of gravity. In the present study, the Flodex apparatus (Hanson Research Corporation, Intrinsic flowability: a new technology for powder-flowability classification, Pharmaceutical Technology, February 1980) was used to measure the granulation flow properties and Flowability Index. The Flodex Operation Manual procedure was used as the experimental method. The Flodex apparatus was set up with the funnel 2 cm above the cylinder assembly. Each set of flow experiments used the 16-mm flow measurement disk as the starting point. Approximately 50 grams of granules were carefully loaded into the cylindrical container to prevent packing. Thirty seconds after all the granulation had been added to the cylinder the release lever was slowly actuated allowing the closure plate to open. If the material flowed through the 16-mm disk, smaller diameter flow measurement disks were used sequentially until a negative result was obtained. The test was considered to be negative if a sufficient amount of material did not flow through the orifice such that a hole at the bottom of the disk was visible from the top of the cylinder. If the material did not flow through the 16-mm disk, larger diameter flow measurement disks were used sequentially until a positive result was obtained and a visible hole was observed from the top of the cylinder. The Flowability Index is the diameter of the smallest opening through which the granules form a visible hole on three consecutive tests.

The powder flowability was assessed by measurement of bulk/tapped densities and flowability index. Carr index and Hausner ratio, which are commonly used to indicate powder flowability per USP <1174> (see Table 8), were calculated from measured bulk/tapped densities. Additionally, the flowability index and internal-friction coefficient were calculated for the powder formulations. The flowability index is the size of the smallest hole the powder flows through, using a scale of 4-40 mm. The internal-friction coefficient (dynes/cm²) is a measurement of the intrinsic flowability of a powder, which is the ability to flow evenly under the action of gravity and other forces. Measurement of the powder's ability to fall freely through a hole in a plate using Flodex™ test instrument takes into account the interparticle friction affected by particle size, shape, bulk density, porosity, electrostatic charge, cohesion forces, etc. See Hanson et al. The internal-friction coefficient is an indicator of flowability because the weight of the cylinder of powder that is compelled to fall must be greater than the friction on the side surface of the cylinder itself:

K≤490rρ

where

r=radius of the smallest hole that allows the powder to flow freely (cm)

ρ=bulk density of powder (g/mL)

K=internal-friction coefficient (dynes/cm²).

It can also be said that a powder having friction coefficient K and bulk density of ρ will fall freely if

r²≥K/490ρ

TABLE 8
Scale of flowability (from USP <1174>).

| Compressibility/Carr Index | | Hausner Ratio |
|---|---|---|
| C = 100 × p_{tapped} − p_{bulk})/p_{tapped} | Flow Character | H = p_{tapped} /p_{bulk} |
| ≤10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 31-37 | Very Poor | 1.46-1.59 |
| >38 | Very, very poor | >1.60 |

The acid-free and acetic acid-containing fosmetpantotenate:MCC granulation samples showed similar Carr index (˜25) and Hausner ratios (ρ_{tapped}/ρ_{bull}, ˜1.3), which indicates passable powder flowability (Table 9). Although the differences in bulk/tapped densities are not significant, the presence of the fosmetpantotenate on MCC resulted in higher flowability index and friction coefficient K (Table 9).

TABLE 9
Bulk/tapped densities and flowability index of fosmetpantotenate granules.

| | Bulk | Tapped | | | | |
|---|---|---|---|---|---|---|
| | Density | Density | Carr | Hausner | Flow | |
| Sample | (g/mL) | (g/mL) | Index | Ratio | Index | K (cp) |
| 20% | 0.37 | 0.49 | 24.5 | 1.32 | 20 | 18130 |
| fosmetpantotenate: | | | | | | |
| MCC, no acid | | | | | | |
| 20% | 0.36 | 0.48 | 25.0 | 1.33 | 22 | 19404 |
| fosmetpantotenate: | | | | | | |
| MCC, 8.4% AcOH | | | | | | |
| MCC (Avicel® | 0.32 | 0.41 | 22.0 | 1.28 | 8 | 6272 |
| PH200), sieved | | | | | | |

### Stability

For the stability analysis, samples of fosmetpantotenate were subdivided and transferred into separate 20 mL clear scintillation vials (about 1 g sample per vial). Closed vials were then placed individually into 5″×7″ foil pouches (CADPAK N-HD, WVTR 0.0005 g/100 in²/day at 90% RH 40° C.). followed by heat sealing the pouches without desiccant inside. Fosmetpantotenate powder samples were weighed (3 g) and placed directly into the same foil pouches described above, and heat sealed without desiccant inside. Closed samples were stored at 30° C./65% RH and measured after 0, 2, 4, and 8 weeks.

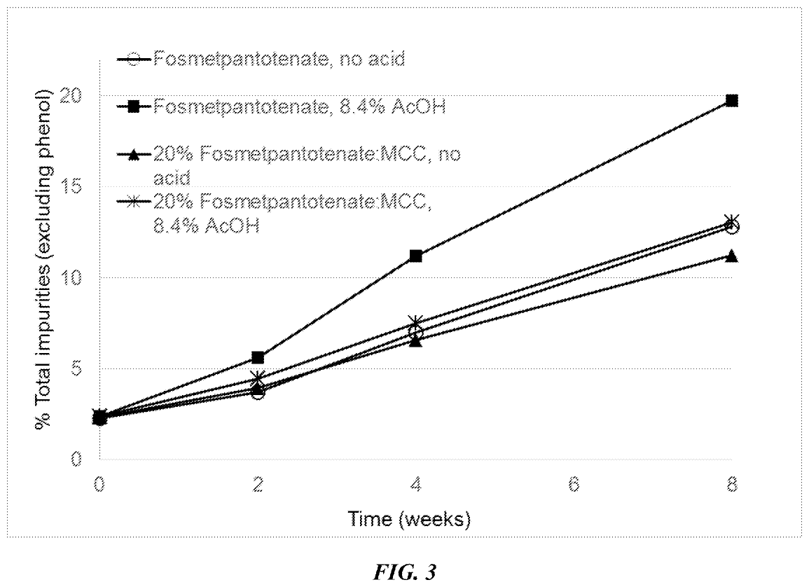

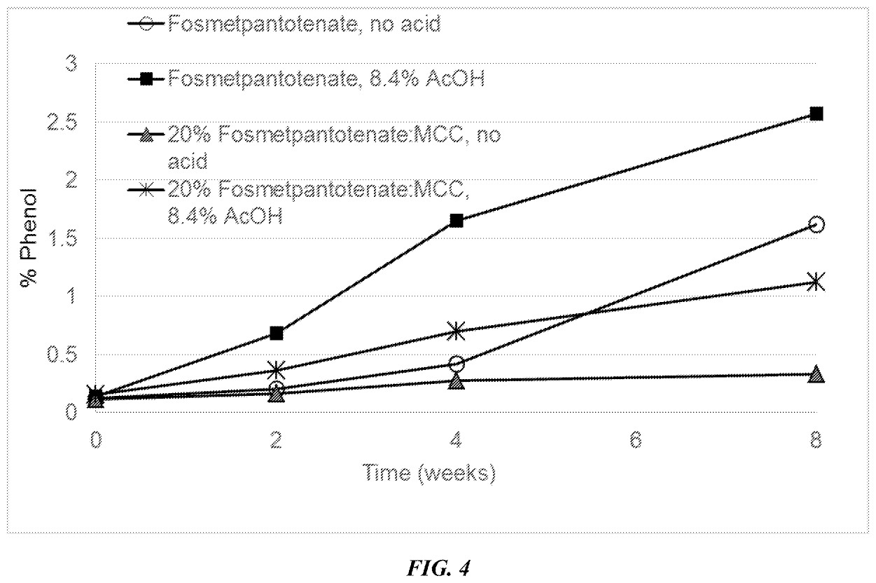

Measurement of impurities was performed by reverse-phase HPLC, utilizing the methods in Table 10. Water:acetonitrile 95:5 (v:v) with 0.005% phosphoric acid was used to extract the API (fosmetpantotenate) from the granulation at concentration of 0.75 mg/mL on an active basis. The sample suspension was ultrasonicated for 10 minutes to ensure complete API extraction followed by filtration using 0.45 μm PTFE filters to remove insoluble materials (i.e., MCC in the granulation samples). The filtrates were then transferred to individual vials and analyzed by HPLC. The results are shown in Tables 11-13 and FIGS. 3 and 4.

TABLE 10
HPLC settings.

| Parameter | Value | | |
|---|---|---|---|
| Column | Waters Atlantis T3, 3.0 μm, 4.6 × 150 mm, PN: | | |
| | 186003729 | | |
| Mobile Phase A | 0.05% Phosphoric acid in water | | |
| Mobile Phase B | 0.05% Phosphoric acid in acetonitrile | | |
| Flow Rate | 1.40 mL/min | | |
| Column Temperature | 30° C. | | |
| Sample Temperature | 5° C. | | |
| Injection Volume | 20.00 μL | | |
| Detection Wavelength | 210 nm (BW: 5 nm, Ref: 380 nm) | | |
| Run Time | 50 min | | |
| Diluent | 0.005% Phosphoric acid in water:acetonitrile 95:5 | | |
| | (v:v) | | |
| | Gradient | | |
| | Time | % MPA | % MPB |
| Program Type | 0.0 | 100 | 0 |
| | 32.0 | 50 | 50 |
| | 40.0 | 50 | 50 |
| | 46.0 | 100 | 0 |
| | 50.0 | 100 | 0 |
| | 0.0 | 100 | 0 |

TABLE 11
Chemical stability for fosmetpantotenate formulations after storage
at 30° C./65% RH for 0, 2, 4, and 8 weeks, as measured by
total impurities (weight percent).

| | Total impurities (%) | | | |
|---|---|---|---|---|
| Formulation | t = 0 | t = 2 wk | t = 4 wk | t = 8 wk |
| Fosmetpantotenate, no | 2.75 | 4.44 | 8.54 | 17.73 |
| acid | | | | |
| Fosmetpantotenate, 8.4% | 2.91 | 8.01 | 16.26 | 25.95 |
| AcOH | | | | |
| 20% Fosmetpantotenate: | 2.75 | 4.56 | 7.56 | 12.35 |
| MCC, no acid | | | | |
| 20% Fosmetpantotenate: | 2.97 | 5.80 | 10.09 | 16.46 |
| MCC, 8.4% AcOH | | | | |

TABLE 12.
Amount of main degradant phenol (as weight percent) after storage
at 30° C./65% RH for 0, 2, 4, and 8 weeks.

| | Phenol (%) | | | |
|---|---|---|---|---|
| Formulation | t = 0 | t = 2 wk | t = 4 wk | t = 8 wk |
| Fosmetpantotenate, no | 0.12 | 0.20 | 0.42 | 1.62 |
| acid | | | | |
| Fosmetpantotenate, 8.4% | 0.14 | 0.68 | 1.65 | 2.57 |
| AcOH | | | | |
| 20% Fosmetpantotenate: | 0.11 | 0.16 | 0.27 | 0.33 |
| MCC, no acid | | | | |
| 20% Fosmetpantotenate: | 0.15 | 0.36 | 0.70 | 1.12 |
| MCC, 8.4% AcOH | | | | |

TABLE 13
Amount of total impurities, excluding phenol, (as weight percent) after
storage at 30° C./65% RH for 0, 2, 4, and 8 weeks.

| | Impurities, excluding phenol (%) | | | |
|---|---|---|---|---|
| Formulation | t = 0 | t = 2 wk | t = 4 wk | t = 8 wk |
| Fosmetpantotenate, no | 2.28 | 3.69 | 7.01 | 12.82 |
| acid | | | | |
| Fosmetpantotenate, 8.4% | 2.37 | 5.60 | 11.19 | 19.73 |
| AcOH | | | | |
| 20% Fosmetpantotenate: | 2.32 | 3.95 | 6.58 | 11.25 |
| MCC, no acid | | | | |
| 20% Fosmetpantotenate: | 2.38 | 4.46 | 7.50 | 13.02 |
| MCC, 8.4% AcOH | | | | |

These data suggest that MCC granulation improves stability relative to fosmetpantotenate alone. Additionally, the absence of acetic acid was found to improve the stability of fosmetpantotenate, alone and when formulated as an MCC granulation.

All of the U.S. patents, U.S. patent application publications, U.S. patent applications, foreign patents, foreign patent applications, and non-patent publications referred to in this specification and/or listed in the Application Data Sheet, including U.S. Provisional Patent Application No. 62/456,077 filed on Feb. 7, 2017 and U.S. Provisional Patent Application No. 62/488,618 filed on Apr. 21, 2017, are incorporated herein by reference, in their entirety.

While specific embodiments of the invention have been illustrated and described, it will be readily appreciated that the various embodiments described above can be combined to provide further embodiments, and that various changes can be made therein without departing from the spirit and scope of the invention. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A solid pharmaceutical formulation comprising (a) a pharmaceutically acceptable solid excipient, and (b) a compound of formula I: 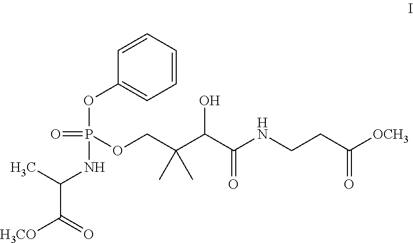 [SMILES: COC(=O)CCNC(=O)C(O)C(C)(C)COP(=O)(NC(C)C(=O)OC)Oc1ccccc1]
or a pharmaceutically acceptable salt thereof.

2. The formulation according to claim 1, wherein said compound of formula I has the following structure:

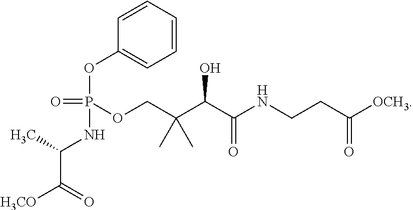 [SMILES: COC(=O)CCNC(=O)[C@H](O)C(C)(C)COP(=O)(N[C@@H](C)C(=O)OC)Oc1ccccc1]

3. The formulation according to any one of the preceding claims, wherein said excipient has a nominal particle size of greater than or equal to 100 μm.

4. The formulation according to claim 3, wherein said excipient has a nominal particle size of greater than or equal to 200 μm.

5. The formulation according to claim 4, wherein said excipient has a nominal particle size of greater than or equal to 300 μm.

6. The formulation according to any one of the preceding claims, wherein said excipient has a pore volume of greater than or equal to 0.01 cm³/g.

7. The formulation according to claim 6, wherein said excipient has a pore volume of greater than or equal to 0.1 cm³/g.

8. The formulation according to claim 7, wherein said excipient has a pore volume of greater than or equal to 1.0 cm³/g.

9. The formulation according to claim 8, wherein said excipient has a pore volume of greater than or equal to 3.0 cm³/g.

10. The formulation according to any one of the preceding claims, wherein said excipient has a specific surface area greater than or equal to 0.5 m²/g.

11. The formulation according to claim 10, wherein said excipient has a specific surface area greater than or equal to 100 m²/g.

12. The formulation according to claim 11, wherein said excipient has a specific surface area greater than or equal to 200 m²/g.

13. The formulation according to claim 12, wherein said excipient has a specific surface area greater than or equal to 300 m²/g.

14. The formulation according to any one of the preceding claims, wherein said excipient is selected from the group of microcrystalline cellulose, lactose, calcium hydrogen phosphate, croscarmellose sodium, crosslinked polyvinylpyrrolidine, magnesium stearate, sodium stearyl fumarate, starch 1500, xanthan gum, guar gum, sucralose, gelatin, magnesium aluminometasilicate, hydroxypropyl methylcellulose acetate succinate, hydroxypropyl-β-cyclodextrin, mesoporous silica, and mannitol.

15. The formulation according to any one of claims 1-13, wherein said excipient comprises lactose.

16. The formulation according to any one of claims 1-13, wherein said excipient comprises lactose monohydrate.

17. The formulation according to any one of claims 1-13, wherein said excipient comprises mannitol.

18. The formulation according to any one of claims 1-13, wherein said excipient comprises microcrystalline cellulose.

19. The formulation according to any one of the preceding claims, wherein said excipient is at least slightly soluble in water at room temperature.

20. The formulation according to claim 19, wherein said excipient is at least sparingly soluble in water at room temperature.

21. The formulation according to claim 19, wherein said excipient is at least soluble in water at room temperature.

22. The formulation according to claim 19, wherein said excipient is at least freely soluble in water at room temperature.

23. The formulation according to claim 19, wherein said excipient is at least very soluble in water at room temperature.

24. The formulation according to any one of the preceding claims, wherein said compound comprises from 5% to 80% by weight of said formulation.

25. The formulation according to any one of claims 1-23, wherein said compound comprises from 10% to 70% by weight of said formulation.

26. The formulation according to any one of claims 1-23, wherein said compound comprises from 20% to 50% by weight of said formulation.

27. The formulation according to any one of claims 1-23, wherein said compound comprises from 20% to 30% by weight of said formulation.

28. The formulation according to any one of claims 1-23, wherein said compound comprises from 10% to 20% by weight of said formulation.

29. The formulation according to any one of the preceding claims, wherein the increase in total impurities measured by HPLC peak integration against the compound, after storage at 30° C. and 60% relative humidity for 4 weeks, is not greater than 15%.

30. The formulation according to any one of claims 1-28, wherein the increase in total impurities measured by HPLC peak integration against the compound, after storage at 30° C. and 60% relative humidity for 4 weeks, is not greater than 10%.

31. The formulation according to any one of claims 1-28, wherein the increase in total impurities measured by HPLC peak integration against the compound, after storage at 30° C. and 60% relative humidity for 4 weeks, is not greater than 5%.

32. The formulation according to any one of the preceding claims, wherein the formulation has a Hausner ratio of from 1.0 to 1.50.

33. The formulation according to any one of claims 1-31, wherein the formulation has a Hausner ratio of from 1.0 to 1.34.

34. The formulation according to any one of claims 1-31, wherein the formulation has a Hausner ratio of from 1.0 to 1.25.

35. The formulation according to any one of the preceding claims, wherein the formulation is in unit dosage form.

36. A formulation according to any one of the preceding claims for use in treating a neurologic disorder.

37. A formulation according to any one of claims 1-35 for use in treating a disorder associated with pantothenate kinase enzyme deficiency.

38. The formulation for use of claim 37, wherein said disorder is pantothenate kinase associated neurodegeneration.

39. The formulation for use of claim 37, wherein said disorder is 4′-phosphopantothenic acid deficiency.

40. The formulation for use of claim 37, wherein said subject exhibits neurodegeneration with brain iron accumulation.

41. The formulation for use of claim 37-40, wherein said subject has a pantothenate kinase gene (PANK) defect.

42. The formulation for use of claim 41, wherein said PANK gene defect comprises a PANK1 gene defect.

43. The formulation for use of claim 41, wherein said PANK gene defect comprises a PANK2 gene defect.

44. The formulation for use of claim 41, wherein said PANK gene defect comprises a PANK3 gene defect.

45. The formulation for use of claim 41, wherein said PANK gene defect comprises a PANK4 gene defect.

46. A formulation according to any one of claims 1-35 for use in treating a subject having a disorder associated with Coenzyme A deficiency.

47. A formulation according to any one of claims 1-35 for use in treating a condition associated with abnormal neuronal function in a subject in need thereof.

48. The formulation for use of claim 47, wherein the condition is Parkinson's disease, dystonia, extrapyramidal effects, dysphagia, rigidity and/or stiffness of limbs, choreoathetosis, tremor, dementia, spasticity, muscle weakness, or seizure.

49. A formulation according to any one of claims 1-35 for use in treating a condition associated with neuronal cell iron accumulation in a subject in need thereof.

50. A formulation according to any one of claims 1-35 for use in treating a subject having neurodegeneration with brain iron accumulation.

51. The formulation for use according to any one of claims 36-50, wherein the formulation is mixed with water for administration.

52. A method of increasing 4′-phosphopantothenic acid production in a subject in need thereof, the method comprising administering to the subject an effective amount of a formulation according to any one of claims 1-35.

53. The method of claim 52, wherein said subject exhibits overexpression of an enzyme for which Coenzyme A is a synthetic precursor.

54. A method of treating a subject having a disorder associated with pantothenate kinase enzyme deficiency comprising administering to a subject in need thereof an effective amount of a formulation according to any one of claims 1-35.

55. The method of claim 54, wherein said disorder is pantothenate kinase-associated neurodegeneration.

56. The method of claim 54, wherein said disorder is 4″-phosphopantothenic acid deficiency.

57. The method of claim 54, wherein said subject exhibits neurodegeneration with brain iron accumulation.

58. The method of any one of claims 54-57, wherein said subject has a pantothenate kinase gene (PANK) defect.

59. The method of claim 58, wherein said PANK gene defect comprises a PANK1 gene defect.

60. The method of claim 58, wherein said PANK gene defect comprises a PANK2 gene defect.

61. The method of claim 58, wherein said PANK gene defect comprises a PANK3 gene defect.

62. The method of claim 58, wherein said PANK gene defect comprises a PANK4 gene defect.

63. A method of treating a subject having a disorder associated with Coenzyme A deficiency comprising administering to a subject in need thereof an effective amount of a formulation according to any one of claims 1-35.

64. A method of treating a condition associated with abnormal neuronal function in a subject in need thereof, the method comprising administering to the subject an effective amount of a formulation according to any one of claims 1-35.

65. The method of claim 64, wherein the condition is Parkinson's disease, dystonia, extrapyramidal effects, dysphagia, rigidity and/or stiffness of limbs, choreoathetosis, tremor, dementia, spasticity, muscle weakness, or seizure.

66. A method of treating a condition associated with neuronal cell iron accumulation in a subject in need thereof, the method comprising administering to the subject an effective amount of a formulation according to any one of claims 1-35.

67. A method of treating a subject having neurodegeneration with brain iron accumulation, the method comprising administering to the subject an effective amount of a formulation according to any one of claims 1-35.

68. The method according to any one of claims 52-67, wherein administering to the subject an effective amount of the formulation comprises mixing the formulation with water and administering the resulting mixture.
